(19) United States
(12) Patent Application Publication
(10) Pub. No.: US 2026/0020980 A1
(43) Pub. Date: Jan. 22, 2026

(54) HOT ICE CUBE

(71) Applicant: Bassem Alhalabi, P.O. Box 273383, FL (US)

(72) Inventor: Bassem Alhalabi, P.O. Box 273383, FL (US)

(21) Appl. No.: 18/776,214

(22) Filed: Jul. 17, 2024

(51) Int. Cl. A61F 7/02; A61F 7/00; F25B 21/04; H02J 7/00

(52) U.S. Cl. CPC A61F 7/02; F25B 21/04; H02J 7/0042; A61F 2007/0037; A61F 2007/0075; A61F 2007/0087; A61F 2007/0093; A61F 2007/0219; A61F 2007/0244; A61F 2007/0295

(57) ABSTRACT

An electronic instant heat/cool generating cube with wireless recharging capability. It has layers of components, a controller on top which includes temperature adjusting buttons, a rechargeable lithium battery, a layer of thermal insulation, a fan, a heat sink, a Peltier device, and a gel layer on the bottom. The top also includes a wireless charging pad and a USB charging port.

### CROSS-REFERENCE TO RELATED APPLICATIONS

This application has no related applications.

### BACKGROUND OF THE INVENTION

Almost in every household, you find several thermal gadgets which are designed to keep heat or cool for a few minutes for various purposes. A popular example is the heat/cool pouch which is often kept in the freezer to absorb cool and when it is needed, say for a child who just burned his finger, the pouch with so much cool in it will be placed on the affected area to sooth the burning feeling of the affected area. Other people who do not have this pouch would simply use some ice cubes from the freezer. In other cases, if there is an affected skin area when the user desires some warmth, the user cooks the pouch in the microwave to heat it up and then applies it on the skin till the heat dissipates and the user microwaves it again. The Hot/Cold Cube of this invention is to replace the pouch and it give heat or cool instantly by pressing the appropriate button. It is an electronic device, and it should always be kept charged on the wireless charging cradle or plugged into a USB charging source until it is needed

### SUMMARY OF THE INVENTION

In the following description, numerous specific details are set forth to clearly describe the embodiments disclosed herein. However, one skilled in the art will understand that some well-known features have not been described in detail so as not to obscure the invention.

This invention, the Hot-Cold Cube, is an alternative electronic device to the hot/cool pouch. It is a fully integrated self-contained hand-held battery-operated cube, with the top side equipped with wireless charging coil to recharge the battery, and the bottom gel side equipped with heat/cool generating thermoelectric Peltier. Basically, the Hot-Cold Cube is always sitting on a charging cradle to charge, just like any wireless phone charger. When the need arises, say a child burns his finger, the Hot-Cold Cube is instantly picked up from the cradle and its gel side is placed on the burned area of the child's finger. Then the blue button is pressed to generate cool instantly on the gel side by the internal thermoelectric Peltier. The longer you press on the blue button, the cooler the gel side becomes. In the case of other situations, where heat is desired, then the red button is pressed to generate heat on the gel side. The longer you press on the red button, the warmer the gel side becomes. In both cases of heat or cool, both buttons red/blue are used to adjust the temperature up/down. If you push any button twice, the cube will turn off. The cube also features with auto shut-off when it is not in use or when it is placed on the charging cradle.

### BRIEF DESCRIPTION OF DRAWINGS

FIG. 1: the Hot-Cold Cube, shown assembled

FIG. 2: The Hot-Cold Cube of FIG. 1, shown without the cover

FIG. 3: the Hot-Cold Cube of FIG. 1, shown with separation between layers and including cables

FIG. 4: A detailed view of the top layer 210 of the Hot-Cold Cube of FIG. 1

FIG. 5: A detailed view of the lower four layers of the Hot-Cold Cube of FIG. 1

### DETAILED DESCRIPTION OF THE INVENTION

The idea of this invention is built on the concept of the thermoelectric Peltier plates. A Peltier plate is a thin thermal device that operates on DC current where the temperature difference between its two surfaces is proportional to the amount of current. The means, the larger DC current we apply, the larger the temperature deference between the two surfaces. Not only this, but the hot/cold surfaces can be switched by reversing the DC polarity.

Let us say we use a typical Peltier plate capable of 20 differential Fahrenheit degrees, and we label the two sides of the Peltier plate A and B. Now, if we keep the temperature of surface A always equal to the room temperature, then surface B will be 20 degrees higher or lower than the room temperature by 20 degrees, based on the DC current polarity. Also, if we control the amount of the DC current, the differential temperature can be controlled to the desire of the user

So this Hot/Cold Cube invention is basically using a thermoelectric Peltier plate with the temperature of its surface A being always kept equal to the room temperature by means of heatsink and turbo fan. Surface B which bears and temperature deferential is the side that heat-transfer gel and cushion to touch the user's skin. If heat is desired a positive current is injected into the plate, otherwise if cool is desired, a negative DC current tis used. The amount of heat and cool is controlled by the amount if the DC current being injected into the plate.

The DC current is taken from an embedded rechargeable battery, or continuously from a USB cable. The battery is charged by a wireless charging coil or from the USB cable like any smart phone. The DC current direction and intensity are controlled by the microcontroller, all embedded in the self-contained cube. That is our inversion, the Hold-Cold Cube.

In the following description, like reference numbers are used to identify like elements. Furthermore, the drawings are intended to illustrate major features of the exemplary embodiments in a diagrammatic manner. The drawings are not intended to depict every feature of every implementation and are not drawn to scale.

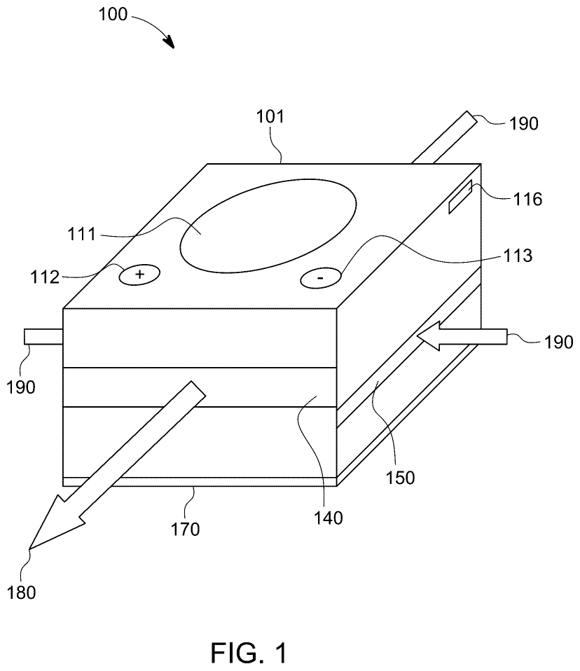

FIG. 1 shows the Hot-Cold Cube 100 as a fully integrated self-contained system. The Hod-Cold Cube 100 is composed with cube cover box 101 which has the following external features:

On the top, the Cube cover box 101 has a wireless charging pad 111, a red switch pad 112, and a blue switch pad 113.

On the right side, there is a USB charging socket 116. On the right, back, and left sides, there are three intake vents 150, each of which permits incoming airflow 190 to enter the cube. On the front, there is one out-take vent 140, through which outgoing airflow 180 is blown out of the cube. The incoming airflow 190 is used to act as a heat source or sink, depending upon the mode of operation of the cube, for the thermoelectric elements, by providing a continuous flow of air at ambient temperature.

On the bottom of the cube, there is a gel layer with cushion surface 170.

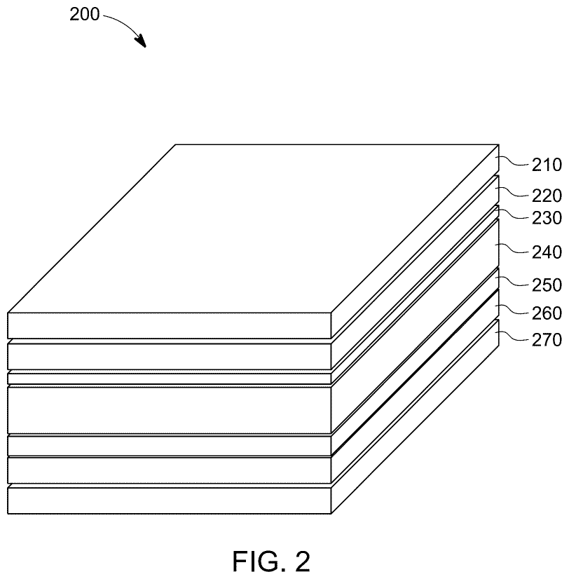

FIG. 2 shows the Hot-Cold Cube 100 with its cover box 101 removed, showing the seven exposed layers of internal components of the cube. These seven layers are:

The first or top layer 210 comprises the main Printed Circuit Board or PCB with all electronics other than the thermoelectric elements on it.

The second layer 220 is a rechargeable lithium battery.

The third layer 230 is a thermal insulation layer, which insulates battery 220 from the remainder of the device.

The fourth layer 240 includes front out-take vent 140 and a turbo fan.

The fifth layer 250 is a heat sink, and includes intake vents 150. The heat sink 250 will transfer heat from the thermoelectric device to the airflow when the cube is in cooling mode, and will transfer heat from the airflow to the thermoelectric device when the cube is in heating mode.

The sixth layer 260 is the thermoelectric device, also called a Peltier device or Seebeck device, which is the component which transfers heat into or out of the gel layer.

The seventh layer 270 is the heat-transfer gel layer, which transfers heat to the patient's skin from the thermoelectric layer or from the patient's skin to the thermoelectric layer. This gel layer includes cushion surface 170, which provides a soft and flexible surface for contact with the patient's skin.

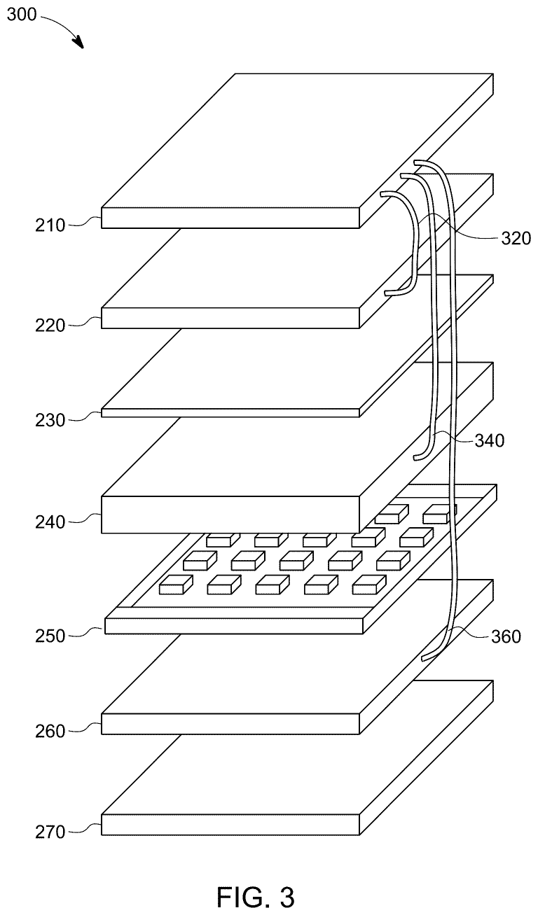

FIG. 3 shows the seven layers of the cube 100 distanced from each other. Also in this figure we see the three cables connecting the first layer 210, which is the printed circuit board and includes the controller, to other components. Cable 320 connects PCB layer 210 to rechargeable lithium battery 220. Cable 340 connects PCB layer 210 to the fan of layer 240. Cable 360 connects PCB layer 210 to the thermoelectric elements of layer 260.

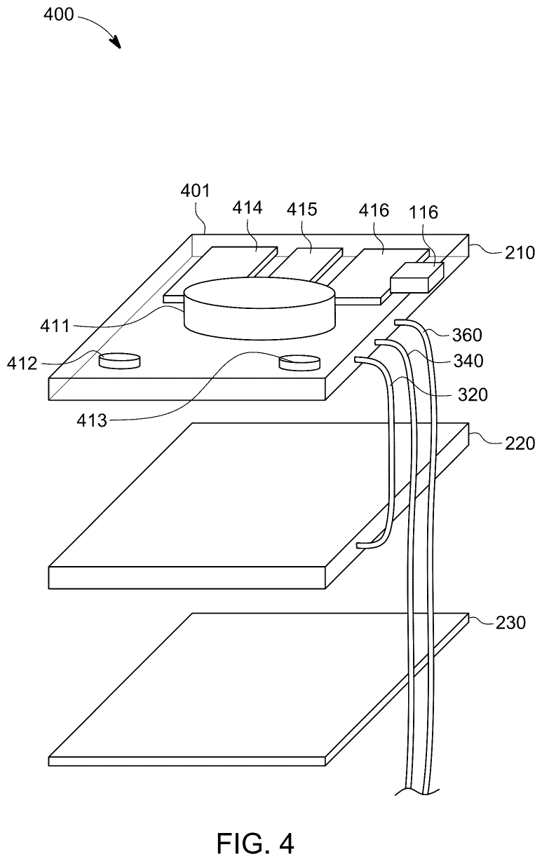

FIG. 4 reveals more details on the first layer 210, which is composed of the following components:

PCB 401, on which the rest of the components are mounted; USB charging socket 116; wireless charging coil 411, which is underneath the wireless charging pad 111 of FIG. 1; red switch 412, which is underneath the red switch pad 112 of FIG. 1; blue switch 413, which is underneath the blue switch pad 113 of FIG. 1; microcontroller 414, which runs the embedded software used to read sensors 415 and the red and blue switch values to determine if the user is requiring heat or cooling and the targeted temperature level; sensor system 415, which provides feedback to the microcontroller 414; and wireless charging circuit 416, which regulates the electric energy coming from coil 411 to charge battery 220 via cable 320.

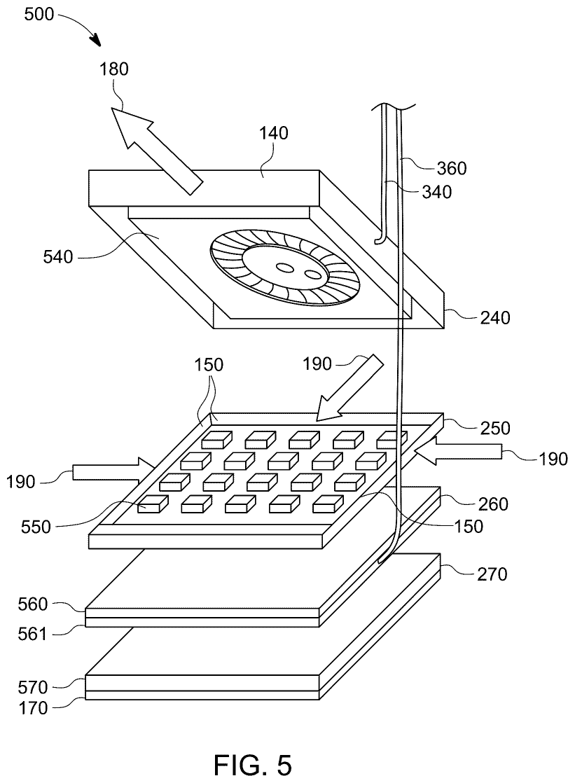

FIG. 5 reveals more details on the lower four layers 240, 250, 260, and 270.

Layer 240 is a compartment composed of turbine fan 540, which is a thin and flat fan, which blows air up from the layer below and out the front of the cube, and out-take vent 140, where the fan air is blown out.

Layer 250 is the heatsink, and composed of the following components: three in-take vents 150 on the right, back, and left sides for intake airflow 190 to flow into the heatsink 250; an array of heat dissipating pillars 550, which take heat from the thermoelectric layer 260 and dissipates it into the airflow as it passes through the heatsink 250 when the cube is operating in cooling mode. When the cube is operating in heating mode, the heat dissipating pillars 550 will take heat from the air and pass it to the thermoelectric layer 260.

Layer 250 is the thermoelectric layer, and has two surfaces; upper surface 260, which is attached to the bottom of heatsink 250, and lower surface 561 which is attached to layer 270.

Layer 270 is the heat-transfer layer, which transfers the heat from the thermoelectric layer's lower surface 561 to the user in heating mode, and transfers heat from the user to the thermoelectric layer's lower surface 561 in cooling mode. Heat transfer layer 270 includes upper surface 570, which is attached to the lower surface 561 of thermoelectric layer 260 and is a heat-conductive gel moldable to the user's skin curves, and lower surface 170, also seen in FIG. 1, which has a heat-conductive cushion surface to provide a smooth touch to the user's skin.

While illustrative embodiments of the invention have been shown and described, variations and alternative embodiments will occur to those skilled in the art. Such variations and alternative embodiments can be made without departing from the scope of the invention as defined in the claims.

As used in this specification and the appended claims, the singular forms “a” and “an” indicate a single element, while “the” may refer back to single or plural referents. Unless defined otherwise, all technical and scientific terms used herein have the same meaning as commonly understood by one of ordinary skill in the art to which the disclosure pertains.

The above detailed description of exemplary and preferred embodiments is presented for the purposes of illustration and disclosure in accordance with the requirements of the law. It is intended to be exemplary but not exhaustive, and is not intended to limit the invention to the precise forms described, but only to enable others skilled in the art to understand how the invention may be suited for a particular use of implementation. No limitation is intended by the description of exemplary embodiments which may have included tolerances, feature dimensions, specific operating conditions, engineering specifications, or the like, and which may vary between implementations or with changes to the state of the art, and no such limitation should be implied therefrom. Applicant has made this disclosure with respect to the current state of the art, but also contemplates advancements and that adaptations in the future may take into consideration those advancements in accordance with the then current state of the art. It is intended that the scope of the invention be defined by the Claims as written and equivalents as applicable. Reference to a claim element in the singular is not intended to mean “one and only one” unless explicitly so stated. No claim element herein is intended to be construed under the provisions of 35 U.S.C. 112 (f), unless the element is expressly recited using the exact phrase “means for . . . ” and no method or process step herein is to be construed under the provisions of 35 U.S.C. section 112 (f) unless the step, or steps, are expressly recited using the exact phrase “step(s) for . . . ”.

1. A self contained electronic thermal differential system in a handheld shape, which is configured to heat or cool the bottom surface such that it can be used to provide heat or cold to a desired area of a user's skin, comprising:
a wireless charging pad;
a first switch pad;
a second switch pad;
a USB charging socket;
three intake vents for air to blow in;
an outtake vent for air to blow out; and
a gel layer.

2. The self contained electronic thermal differential system of claim 1, wherein the components of the self contained electronic thermal differential system are arranged in seven layers, which are, from top down,
first, a PCB layer, which includes a wireless charging coil, a first switch, a second switch, a microcontroller, sensors, a charging control circuit, and a USB charging socket, wherein the microcontroller is configured to control all components using feedback from the sensors and inputs from the switches and charging control circuit;
second, a rechargeable battery, which is configured to be recharged using each of the charging coil and a USB cable;
third, a thermal insulation layer to protect the battery;
fourth, a fan layer, which includes a fan and the air outtake vent;
fifth, a heat sink layer, which includes the three air intake vents and a heat exchanging material;
sixth, a thermoelectric layer, which includes at least one thermoelectric element; and
seventh, a heat-transfer gel layer, which includes a soft cushion configured for use directly on a user's skin.

3. The self contained electronic thermal differential system of claim 2, wherein the microcontroller is configured to control all components using feedback from the sensors and inputs from the switches and charging control circuit by accepting input from the first switch as indicating the user desires a cooler temperature of the gel, by accepting input from the second switch as indicating the user desires a warmer temperature of the gel, and by accepting a pushing of a single button twice as input to turn the cube off;
wherein the controller is further configured to automatically shut off the operation of the thermoelectric layer of the cube in response to each of the cube being placed on the charging cable and the absence of user input for a predetermined period of time indicating that the cube is not in use.
